(19) United States
(12) Patent Application Publication
(10) Pub. No.: US 2026/0174398 A1
(43) Pub. Date: Jun. 25, 2026

(54) METHODS AND SYSTEMS FOR PREDICTING AND PREVENTING OR MINIMIZING THE EXTENT OF INTRAMYOCARDIAL HEMORRHAGE IN REPERFUSED ACUTE MYOCARDIAL INFARCTIONS

(71) Applicant: The Trustees of Indiana University, Bloomington, IN (US)

(72) Inventors: Keyurkumar P. Vora, Bloomington, IN (US); Khalid Youssef, Bloomington, IN (US); Rohan Dharmakumar, Zionsville, IN (US)

(73) Assignee: The Trustees of Indiana University, Bloomington, IN (US)

(21) Appl. No.: 19/133,438

(22) PCT Filed: Nov. 30, 2023
(86) PCT No.: PCT/US2023/081855

§ 371 Date: May 28, 2025

Related U.S. Application Data
(60) Provisional application No. 63/429,001, filed on Nov. 30, 2022.

(51) Int. Cl. A61B 5/00; A61B 5/02; A61B 17/22

(52) U.S. Cl. CPC A61B 5/7275; A61B 5/02007; A61B 2017/22067; A61K 2300/00

(57) ABSTRACT

Processes are provided based on an interventional (device-based) approach to minimize the development of intramyocardial hemorrhage within the myocardial infarction (MI) zone. In one exemplary process, the revascularization occurs in an incremental fashion prior to fully opening the vessel, so as to modulate the hydrostatic pressure transmitted from the proximal end of the catheter, prior to placing a stent. In another exemplary process, the stent will be placed within the central core of the embolization and will slowly expand within the lumen reaching full patency over a period of time. Devices operated with these processes can also greatly reduce the chance of development of intramyocardial hemorrhage with the MI zone. These developments can improve the outcome in hemorrhagic patients, who otherwise carry a significant risk for heart failure and sudden death.

### CROSS-REFERENCE TO RELATED APPLICATIONS

This application includes a claim of priority under 35 U.S.C. § 119(e) to U.S. provisional patent application No. 63/429,001, filed Nov. 30, 2022, the entirety of which is hereby incorporated by reference.

### FIELD OF INVENTION

This invention relates to methods and devices to reduce development of intramyocardial hemorrhage within the myocardial infarction zone.

### BACKGROUND

Acute myocardial infarction (MI) stemming from sudden embolic obstruction of the coronary artery leading to abrupt decrease in blood flow to the heart muscle affects nearly a million people in the US alone. Immediate care of these patients is crucial for preventing sudden death. Accordingly, the current stand-of-care for acute MI is to implement immediate and rapid revascularization of the coronary artery via an introduction of a stent or drugs that can recanalize the artery. It is counter-intuitive, against the stand-of-care, and difficult, if not impossible, to carry out for the cardiac interventionalist to reperfuse acute MI patients slowly, because it is believed that the patient would continue to lose heart muscle due to continued ischemia if slow reperfusion was to be performed.

One of the complications with these procedures is that although large epicardial coronary artery can be opened in this fashion, smaller vessels downstream can remain obstructed (known as microvascular obstruction) or burst (also known as intramyocardial hemorrhage). Intramyocardial hemorrhage is the most significant form of the tissue injury associated with revascularization and is known to occur in 40-70% of the patients undergoing revascularization for acute myocardial infarction. Growing literature continues to outline major complications (infarct expansion, prolonged inflammatory burden, and accelerated adverse remodeling of the heart), all precipitating in accelerated heart failure in patients who suffer hemorrhagic myocardial infarction. Recent studies have also shown that reperfusion hemorrhage is a key predictor of adverse outcomes in the post-infarction period. The occurrence of hemorrhage has been shown to drive a larger infarct size and adverse remodeling in the post-infarction period, both of which are key contributors to the heart failure epidemic.

Yet, there are no known methods to reduce the extent of hemorrhage in the setting of reperfused myocardial infarction.

Therefore, it is an object of the present invention to provide methods or processes to identify the vulnerable subjects and to reduce the incidence and/or extent of hemorrhage post revascularization, thereby minimizing the effects of reperfusion hemorrhage and limiting the acute and chronic cardiac damage.

It is another object of the present invention to provide systems to allow for operation of revascularization devices in a manner that mitigates the incidence and/or extent of hemorrhage.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide methods for treating a subject with myocardial infarction (MI), or performing revascularization in a subject in need thereof (such as a subject with MI, wherein the subject with the MI having at least one occluded coronary blood vessel causing the acute MI), and the methods include: (i) identifying a subject with a likelihood to develop intramyocardial hemorrhage by: (a) determining or obtaining results that the subject has an ongoing inflammation-related disorder, diabetes, hypertension, a metabolic disease, or a combination thereof, or that the subject with MI has had the diabetes, the hypertension, the metabolic disease, or a combination thereof prior to the MI; (b) performing, or obtaining or requesting results of, an angiogram in the subject to determine absence of collateral coronary circulation in the subject; or (c) performing, or obtaining or requesting results of, a full occlusion of an ischemic artery, AND, a ST-segment score of at least 5.5 and/or an absence of collateral coronary circulation of the ischemic artery; OR performing, or obtaining or requesting results that the occlusion of the ischemic artery is only partial and not full occlusion, the ST-segment score is greater than 10, and the collateral coronary circulation of the ischemic artery is present, so as to determine likelihood of intramyocardial hemorrhage following a standard-rate revascularization intervention; or a combination of (a)-(c); and (ii) performing a gradual revascularization intervention in the subject with the MI who is identified with the likelihood to develop intramyocardial hemorrhage, wherein the gradual revascularization intervention is slower or takes longer in restoring myocardial reperfusion compared to the standard-rate vascularization intervention.

In some embodiments, a method is provided for treating a subject with myocardial infarction (MI), or performing revascularization in a subject with MI, the subject with the MI having at least one occluded coronary blood vessel causing the acute MI, and the method includes: performing a standard-rate (or rapid) revascularization intervention to a subject with the MI who is identified as not having a likelihood or lower likelihood to develop intramyocardial hemorrhage, wherein the subject not having the likelihood or lower likelihood is identified by: determining or obtaining results that the subject does not have an ongoing inflammation-related disorder, diabetes, hypertension, or a metabolic disease, and that the subject did not have diabetes, hypertension, or a metabolic disease prior to the MI; performing, or obtaining or requesting results of, an angiogram in the subject to determine presence of collateral coronary circulation in the subject; and/or performing, or obtaining or requesting results of, a partial occlusion of an ischemic artery of the subject and a presence of collateral coronary circulation of the ischemic artery, OR a partial occlusion of the ischemic artery with an absence of the collateral coronary circulation and a ST-segment score of 10 or less, OR a total occlusion of the ischemic artery with a presence of the collateral coronary circulation and a ST-segment score of less than 5.5, to determine an unlikelihood of intramyocardial hemorrhage following a standard-rate revascularization intervention.

Further embodiments include calculating a total score of an assigned ST-segment score point, an assigned occlusion point, and an assigned collateral point of the subject, wherein a higher total score compared to a reference individual who did develop hemorrhage after a revascularization therapy indicates that the subject is likely to develop the hemorrhage.

In other embodiments, the methods include calculating a total score of an assigned ST-segment score point, an assigned occlusion point, and an assigned collateral point of the subject, wherein a lower total score compared to a reference individual who did not develop hemorrhage after a revascularization therapy indicates that the subject is unlikely to develop the hemorrhage; wherein the point assignment includes a higher point for a higher ST-segment score relative to a lower ST-segment score, a higher point for full occlusion relative to partial occlusion, and a higher point for absence of collateral coronary circulation relative to presence of the collateral coronary circulation.

Some embodiments provide a method for treating a subject with myocardial infarction (MI), or performing revascularization in a subject with MI, the subject with the MI having at least one occluded coronary blood vessel causing the acute MI, and the method includes: performing a gradual revascularization intervention in the subject with the MI, wherein the gradual revascularization intervention is slower or takes longer in restoring myocardial reperfusion compared to a standard-rate vascularization intervention.

In some aspects, the subject undergoing or in need of a gradual revascularization intervention has not been identified as having likelihood or unlikelihood of developing hemorrhage. In other aspects, the subject undergoing or in need of a gradual revascularization intervention is one who has been identified with a likelihood to develop intramyocardial hemorrhage by: (a) determining or obtaining results that the subject has an ongoing inflammation-related disorder, diabetes, hypertension, a metabolic disease, or a combination thereof, or that the subject with MI has had the diabetes, the hypertension, the metabolic disease, or a combination thereof prior to the MI; (b) performing, or obtaining or requesting results of, an angiogram in the subject to determine absence of collateral coronary circulation in the subject; or (c) performing, or obtaining or requesting results of, a ST-segment score of greater than 10 from electrocardiography, OR full occlusion of an ischemic artery of the subject, OR an absence of collateral coronary circulation of the ischemic artery with a ST-segment score of at least 5.5, to determine likelihood of intramyocardial hemorrhage following a standard-rate revascularization intervention; or a combination of (a)-(c).

In various aspects, revascularization intervention comprises percutaneous coronary intervention, intracoronary stent, or atherectomy device.

In various aspects, standard-rate revascularization intervention comprises a percutaneous coronary intervention or a cardiac catheterization configured to increase pressure distal to a catheter from substantially zero to systolic pressure, or from a starting pressure to a systolic pressure, of the subject over a duration of no more than 5 minutes or no more than 10 minutes.

In various aspects, a gradual revascularization intervention comprises a percutaneous coronary intervention or a cardiac catheterization configured to increase pressure distal to a catheter or an instrument or stent configured to increase blood flow when placed in blood vessel bed from substantially zero to systolic pressure, or from a starting pressure to a systolic pressure, of the subject over a duration of at least 15 minutes, at least 30 minutes, at least 40 minutes or at least 50 minutes and up to 1 hour.

In various aspects, gradual revascularization intervention comprises a percutaneous coronary intervention or a cardiac catheterization configured to increase pressure distal to a catheter from substantially zero to systolic pressure, or from a starting pressure to a systolic pressure, of the subject over a duration that is at least 100%, at least 200%, or at least 300% longer than that for a standard-rate revascularization intervention.

In various aspects, performing the angiogram further comprises segmenting blood vessels from the angiogram to determine absence or presence of collateral circulation in each segment of the blood vessels. In various aspects, the methods restore TIMI grade 3 flow in the occluded coronary blood vessel in the subject.

Methods are also provided for assessing a subject in need of a coronary revascularization therapy, and/or predicting the likelihood of developing intramyocardial hemorrhage following a standard-rate coronary revascularization therapy in the subject, and the methods include:
performing an electrocardiogram in the subject to obtain ST-segment score, and performing a coronary angiogram in the subject to determine presence or absence of collateral coronary circulation and extent of culprit artery obstruction;
and
determining the subject is likely to develop intramyocardial hemorrhage following the standard-rate coronary revascularization therapy, if:
   (1) the extent of culprit artery obstruction is full occlusion of an ischemic artery, AND, the ST-segment score is at least 5.5 and/or the collateral coronary circulation of the ischemic artery is absent; or
   the extent of culprit artery obstruction is only partial occlusion of the ischemic artery, AND, the ST-segment score is greater than 10 and the collateral coronary circulation of the ischemic artery is absent; or
   (2) a total score of an assigned ST-segment score point, an assigned occlusion point, and an assigned collateral point, is higher than that of a reference individual who did develop hemorrhage after the standard-rate revascularization therapy;
or determining the subject is unlikely or has a reduced likelihood to develop intramyocardial hemorrhage following the standard-rate coronary revascularization therapy, if:
   (3) the extent of culprit artery occlusion is only partial occlusion and the collateral coronary circulation is present; or
   the extent of culprit artery occlusion is only partial occlusion, the collateral coronary circulation is absent, and the ST-segment score is 10 or less; or
   the extent of culprit artery occlusion is full occlusion of the ischemic artery, the collateral coronary circulation is present, and the ST-segment score is less than 5.5; or
   (4) the total score of the assigned ST-segment score point, the assigned occlusion point, and the assigned collateral point, is lower than that of a reference individual who did not develop hemorrhage after the standard-rate revascularization therapy;
wherein the point assignment includes a higher point for a higher ST-segment score relative to a lower ST-segment score, a higher point for full occlusion relative to partial occlusion, and a higher point for absence of collateral coronary circulation relative to presence of the collateral coronary circulation, for a same individual.

In various aspects, the assessment/prognosis methods further include prescribing or administering the standard-rate revascularization therapy to the subject determined to have an unlikelihood or reduced likelihood of developing the hemorrhage.

In various aspects, the assessment/prognosis methods further include prescribing or administering a gradual revascularization intervention to the subject determined to have an likelihood of developing the hemorrhage.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A shows representative cases of pre-PCI CAG images for collateral grading by Rentrop's Classification. FIG. 1B shows infarct size (left) and hemorrhage volume (right) of hemorrhagic MI cases with and without collaterals. FIG. 1C shows representative cases with and without collaterals as observed from emergency.

FIG. 2 depicts in panel a), superposable neural-network comparison to conventional deep neural-network (Prior Art); in panel b), individual feature-functions, wherein the exact function of each feature as given by the SNN, where the final outcome is the sum of all feature-functions; and in panel c), Simplified predictive model of post-reperfusion intramyocardial hemorrhage (IMH), wherein with approximate accuracy, sensitivity, and specificity of 80%, the model requires three features: presence/absence of collaterals, total/partial culprit artery obstruction, and ST-segment score. The outcome is determined by mapping the ST-segment score to the appropriate curve, based on the presence or absence of collaterals, and whether the culprit artery is partially or totally obstructed. In some embodiments, the curve for the approximation of the function of the “ST-segment score” is an exponential function: ae^{(bχ}^{₃}⁾+c, where χ₃ is the ST-segment score. In further embodiments, the other two features (total/partial vessel obstruction and presence/absence of collateral circulation) are binary, e.g., they each carry a value of 0 or 1, and thus are not represented by a continuous curve. The functions of these features are given graphically in FIG. 2 panel b).

FIG. 3 depicts that a gradual reperfusion following 3 hours of LAD-ligation in an animal reduces hemorrhage and infarct size (Animal #2) compared to an animal (Animal #1) that was reperfused immediately (standard reperfusion) to the full extent possible after the same 3-hour ischemic time. In the short-axis T2* images, the bright thick dashed lines demarcate the zone hemorrhage; and in LGE CMR, the thick dashed lines demarcate the infarct zone. The chart details hemorrhage volume and infarct size in Animal #1 and Animal #2.

FIG. 4 depicts an exemplary point system for determining likelihood of developing intramyocardial hemorrhage based on ST-segment score, condition of blood vessel obstruction, and presence or absence of collateral circulation, before a revascularization therapy.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

Percutaneous coronary intervention (PCI) is a revascularization procedure that uses a catheter (or a thin flexible tube) to place a small structure (such as a stent) to open up blood vessels that have been narrowed by plaque buildup or blockage due to an embolus/thrombus. Initially PCI referred to mostly percutaneous transluminal coronary angioplasty (PTCA); and currently, rotational atherectomy, directional atherectomy, extraction atherectomy, laser angioplasty, implantation of intracoronary stents and other catheter devices for treating obstructive coronary artery disease, such as acute myocardial infarction (MI), unstable angina, and multivessel coronary artery disease (CAD), are considered components of PCI. Standard guidelines for performing PCI are described in references such as J. S. Lawton et al., Circulation. 2022; 145:e18-e114 and S. C. Smith Jr. et al., Circulation. 2001; 103:3019-3041.

An angiogram, also known as an arteriogram, is an X-ray of the arteries and veins, used to detect blockage or narrowing of the vessels.

A subject, patient, or individual (used interchangeably) can be one who has been previously diagnosed with or identified as suffering from or having a disease-state (e.g., myocardial infarction) in need of monitoring or one or more complications related to such a disease-state, and optionally, have already undergone treatment for the disease-state or the one or more complications related to the disease/condition. A “subject” means a human or animal. Usually the animal is a vertebrate such as a primate, rodent, domestic animal or game animal. In various embodiments, the subject is a human.

Collateral circulation, also denoted as collaterals, typically refers to vessels sprouting from non-culprit coronary arteries. Collaterals can be identified manually or through machine-learning algorithms applied to the coronary angiograms. In some aspects, angiograms are segmented to assess presence of collateral circulation.

Thrombolysis in myocardial infarction (TIMI) coronary flow grading system records epicardial perfusion, typically on coronary arteriography. TIMI grading system has the following grades: Grade 0 means complete occlusion of the infarct-related artery; Grade 1 means some penetration of contrast material beyond the point of obstruction but without perfusion of the distal coronary bed; Grade 2 means perfusion of the entire infarct vessel into the distal bed but with delayed flow compared with a normal artery; and Grade 3 means full perfusion of the infarct vessel with normal flow.

Total culprit artery obstruction, as well as partial culprit artery obstruction, can be determined via coronary angiograms determined using X-ray imaging systems. “Culprit artery” or “culprit coronary artery” generally refers to any vessel with an acute thrombotic total or subtotal occlusion.

A normal ECG contains waves, intervals, segments and one complex. Wave: A positive or negative deflection from baseline that indicates a specific electrical event corresponding to the cardiac cycle event of systole and diastole. The waves on an ECG include the P wave, Q wave, R wave, S wave, T wave and U wave. Interval: The time between two specific ECG events. The intervals commonly measured on an ECG include the PR interval, QRS interval (also called QRS duration), QT interval and RR interval. Segment: The length between two specific points on an ECG that are supposed to be at the baseline amplitude (not negative or positive). The segments on an ECG include the PR segment, ST segment and TP segment. Complex: This is a combination of multiple waves grouped together. The only main complex on an ECG is the QRS complex. Point: There is only one point on an ECG termed the J point, which is where the QRS complex ends and the ST segment begins.

ST segment represents the interval between depolarization and repolarization of the ventricles. It can be evaluated by using as baseline reference both the PQ and the TP segments, which are both expression of the diastolic potentials. A ST-segment score can be determined by a cardiologist by examining the electrocardiogram (ECG) using a standard approach that measures the elevation of the ST segment peak from the baseline where there's no electrical activity. In various aspects, ST-score calculation is done by systematic analysis of standard 12-lead electrocardiogram (12-lead ECG). Standard ECG contains 12 leads including six limb leads (I, II, III, aVL, aVR and aVF) and precordial leads (V1, V2, V3, V4, V5 and V6). The main part of an ECG contains a P wave, QRS complex and T wave. ST score calculation begins with the identification of the J-point in each lead, representing the junction between the termination of the QRS complex and the onset of the ST-segment. The baseline segment is TP segment. Normal ECG shows horizontal ST-segment without any deviation in relation to baseline and thus normal ECG ST-score is 0. However, in case of AMI, ST-segment deviation is the basis of diagnosis. This deviation, measured in millimeters, is then quantified at the J-point, considering both polarity (elevation or depression) and amplitude. These individual scores are then summed to calculate the total ST-segment score, providing an overall assessment of the magnitude of myocardial ischemia.

The term “about” or “approximately” when used in connection with a referenced numeric indication (in percentage) means the referenced numeric indication (in percentage) plus or minus up to 5% of that referenced numeric indication (in percentage), unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Various embodiments of this invention provide for a process to overcome the development of hemorrhage within the infarction by altering the reperfusion conditions. In various implementations, the methods or processes to mitigate development of hemorrhage include two steps: (i) Identifying vulnerable patient; and (ii) Performing reperfusion at an effectively slow speed or gradual fashion, so as to revascularize with minimal (or absence at best) of development of hemorrhage.

Step i—Identification of Vulnerable Patient:

Various embodiments provide that Step i includes one or more of:
(a) obtaining medical history of a patient to determine gender, or presence of diabetes, hypertension, metabolic diseases, smoking or any ongoing inflammatory process, so as to assess/indicate likelihood of developing myocardial hemorrhage;
(b) identifying absence or presence of collateral circulation by performing or obtaining a result of a coronary angiogram in the subject, prior to performing angioplasty in the subject, so as to assess/indicate likelihood or unlikelihood (or lower likelihood) of developing myocardial hemorrhage, respectively; that is, absence of collateral circulation indicates that the subject is more likely than not to develop myocardial hemorrhage with PCI or other reperfusion therapy, whereas having collateral circulation indicates that the subject is at a reduced risk, or in some instances unlikely, to develop myocardial hemorrhage with PCI or other revascularization therapy; and
(c) performing one or more computer-implemented simulations (or modeling) of hemodynamic parameters of a patient, wherein optionally the hemodynamic parameters include time since chest pain, immediate blood pressure prior to revascularization, metabolic demands, vascular anatomy, and/or location of the embolus;

Medical History—Hemorrhage is a vascular phenomenon and as such potential factors that compromise the vascular integrity (diabetes, hypertension, metabolic diseases, cancer, cirrhosis, hypercholesterolemia or any ongoing inflammatory process) can predispose acute MI patients towards development of hemorrhage during or immediately after reperfusion compared to those that are free of these pre-existing conditions. This may be established through the assessment of patient's medical history. In various aspects, a male individual is more prone to hemorrhage following revascularization therapy to myocardial infarction than a female individual. In some instances, an individual with history of diabetes, hypertension, metabolic diseases, or smoking is more prone to hemorrhage following revascularization therapy than an individual without such history. In some instances, an individual with an ongoing inflammatory process, diabetes, hypertension, or metabolic diseases is more prone to hemorrhage following revascularization therapy than an individual without the presence of these indications. Thus, an individual more prone to hemorrhage is thereby, in some instances, identified as being likely to develop hemorrhage, and thus may be revascularize in a gradual manner. In several instances, the interventionalist moves forward with informed reperfusion prior to intervening in the patient. That is, intervening in the current setting to effectuate the placement of a stent to ensure blood flow is established to a block vessel.

Invasive measurements—A more refined identification involves the determination of patients with collateral circulation (also referred to as collaterals) as they would have diminished risk of developing hemorrhage. This is because the presence of collaterals ensures that downstream microvascular vascular ischemia is reduced as the collaterals (vessels sprouting from non-culprit coronary arteries) continue to supply blood to the region of the heart muscle impacted by the blocked artery. Hence identification of patients without collaterals is crucial since it is these patients that have a very high likelihood of developing hemorrhage. In various aspects, a method includes identifying patients through rapid assessment of collaterals based on the angiograms ascertained immediately prior to angioplasty. Collaterals can be identified manually or through machine-learning algorithms applied to the coronary angiograms. Standard angiograms are known in the art. In some aspects, angiograms are segmented to assess presence of collateral circulation.

Computational modeling of hemodynamics—In addition to the identification of collateral circulation, hemodynamic models to predict the pressure distribution across the culprit artery can be used to predict the vulnerable patient. In various embodiments this includes immediate pressure at revascularization, metabolic demands, vascular anatomy, location of the embolic obstruction. Common values used include but are not limited to pressure prior to revascularization: systolic blood pressure at 80-100 mmHg; metabolic demands: ischemia lasting longer than 60 minutes; vascular anatomy: proximal portions of the coronary artery tree without collateral circulation; location of the embolus: left-anterior descending coronary artery; and having these features indicate that the subject is likely to develop hemorrhage following revascularization therapy. Such a model can be used to fine tune how to effectively revascularize the culprit coronary artery without abrupt changes in flow. In some aspects, a microvascular network fluid dynamics model is used in a computer-implemented simulation, which is based on the relationship between flow and pressure in each segment and the principle of conservation of mass at each bifurcation. See Gruionu G, et al., Am J Physiol Heart Circ Physiol 2005; 288:H2047-2054; Gruionu G, et al., Microcirculation 2012; 19:610-8; Gruionu G, et al., Am J Physiol Heart Circ Physiol 2005; 288:H2778-2784; and Gruionu G, et al., Anat Histol Embryol 2000; 29:31-6. For example, a model estimates the hemodynamic parameters (e.g., intravascular pressure, segment flow rate, and wall shear stress) in the vasculature network (based on the measured morphometric data), and the network can be represented as a series of cylindrical segments, with the bifurcation points as nodes.

In some embodiments, a combination of presence/absence of collaterals, total/partial culprit artery obstruction, and ST-segment score is used collectively to predict a vulnerable (hemorrhage-prone) patient. In some embodiments, partial or total blood vessel obstruction is determined by catheter coronary angiography. In further aspects, a ST-segment score is considered along with the presence or absence of collaterals and whether the culprit artery is partially or totally obstructed in determining likelihood or predicting a vulnerable (hemorrhage-prone) patient. In various embodiments, the total score as a summation of a ST-segment score point, an obstruction point, and a collateral point is computed to determine an individual's likelihood to develop hemorrhage after revascularization therapy. While the assignment of an actual points may be arbitrary or user-determined, generally the greater the total point is, the more likely the individual is to develop hemorrhage after revascularization therapy; and the ST-segment score point is assigned a greater number if the ST-segment score is higher relative to a lower ST-segment score, the obstruction point is assigned a greater number if full obstruction is present relative to partial obstruction, and the collateral point is assigned a greater number if collateral circulation is absent as opposed to present. In some embodiments, a ST-segment score point is 0 if the ST-segment score of the individual's (prior to revascularization therapy) is smaller than 5.5; a ST-segment score point is 1 if the ST-segment score is between 5.5 and 10 (inclusive); and a ST-segment score point is 2 if the ST-segment score is greater than 10; AND an obstruction point is 2 if full obstruction is present, or 0 if full obstruction is absent or not detectable; AND a collateral point is 0 if collateral circulation is present in the individual (prior to revascularization therapy), or 1 if collateral circulation is absent or not detectable; so that if the total score of all three points exceeds 2 (i.e., 3 or greater), then the individual is determined as hemorrhagic prone, or if the total does not exceed 2 (i.e., 2 or less), then the individual is determined as non-hemorrhagic prone.

In other embodiments, if a ST-segment score point, an obstruction point, and a collateral point, and a total of the three of an individual are higher than those of a control subject who develops hemorrhage after revascularization therapy (e.g., standard-rate or rapid revascularization therapy) indicates that the individual would be likely to develop hemorrhage as well under the same revascularization therapy, wherein a same point assignment criterion is used for the individual and the control subject, said criterion comprising: the ST-segment score point is assigned a greater number if the ST-segment score is higher relative to a lower ST-segment score, the obstruction point is assigned a greater number if full obstruction is present relative to partial obstruction, and the collateral point is assigned a greater number if collateral circulation is absent as opposed to present.

In another embodiment, if a ST-segment score point, an obstruction point, and a collateral point, and a total of the three of an individual are smaller than those of a control subject who does not develop hemorrhage after revascularization therapy (e.g., standard-rate or rapid revascularization therapy) indicates that the individual is also unlikely to develop hemorrhage under the revascularization therapy, wherein a same point assignment criterion is used for the individual and the control subject, said criterion comprising: the ST-segment score point is assigned a greater number if the ST-segment score is higher relative to a lower ST-segment score, the obstruction point is assigned a greater number if full obstruction is present relative to partial obstruction, and the collateral point is assigned a greater number if collateral circulation is absent as opposed to present.

In additional aspects, location of site of obstruction relative to the origin or the heart is an additional feature in predicting vulnerability or risk to develop hemorrhage after standard-rate revascularization therapy. In other aspects, location of site of obstruction relative to the heart is an alternative feature, in place of full/partial occlusion in predicting vulnerability or risk to develop hemorrhage after standard-rate revascularization therapy. Typically proximal obstruction is at a much higher risk than a non-proximal location of obstruction; and hence a proximal obstruction point is assigned a greater number than a non-proximal obstruction, for the calculation of a total score. The proximal portion of an artery is closer to its origin or the heart, while the distal portion is farther away. Arbitrarily, coronary artery length is divided into 3 parts and first ⅓ part is considered proximal which is closure to its origin from aorta and because it's proximal, a large area of heart muscle is exposed to infarction.

Superposable neural-network (SNN) modeling—an additive artificial neural network (ANN) optimization framework with dataset division and outcome interpretation techniques—has identified three features: presence/absence of collaterals, total/partial culprit artery obstruction, and ST-segment score, collectively, as sufficient features in predicting post-reperfusion intramyocardial hemorrhage likelihood as exemplified by the score system described above. In various aspects, a SNN framework utilizes a hybrid of model extraction methods and feature-based methods to generate a fully interpretable additive ANN model while simultaneously pruning features and feature interdependencies that are redundant or suboptimal to model performance and generalizability. Further description of SNN framework is provided by Youssef et al. in Communications Earth & Environment volume 4, Article number: 162 (2023). In some aspects, the mathematical representation of the SNN is defined by:

$${S}_{t} ( \{ {χ}_{j} \} ) = {\sum }_{j} ( {\sum }_{k} {w}_{j , k} {e}^{- {( {a}_{j , kχj} + {b}_{j , k} )}^{2} +} {c}_{j} )$$

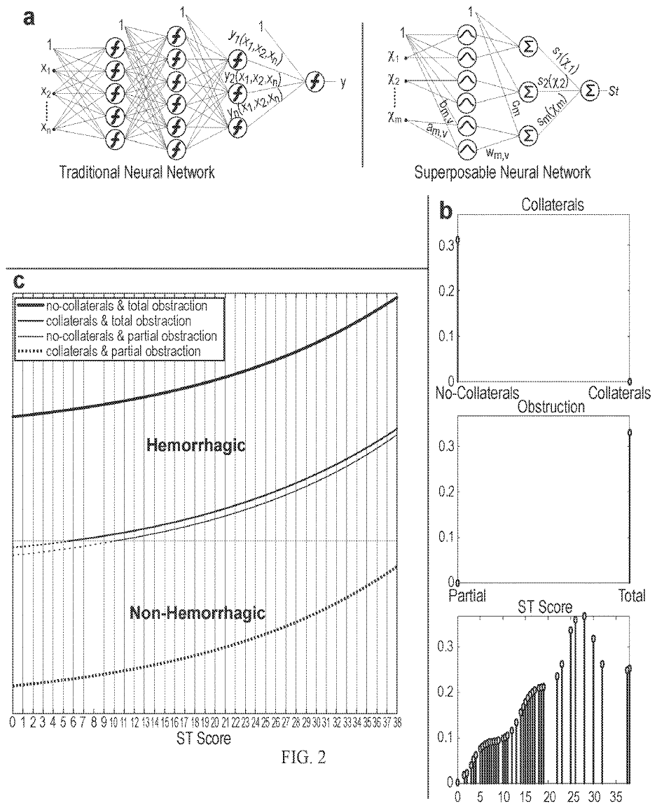

The parameters in this equation correspond to the SNN parameters shown in FIG. 2 panel a); and the function of each feature χⱼ=Σₖw_{j,k}e^{-(a}^{_{j,kχj}}^{+b}^{_{j,k}}⁾^{²}⁺cⱼ and Sₜ({χⱼ}) is the SNN output, which is the sum of all the feature functions.

In actions (b) and (c) of Step i, a patient in need of or being intervened can be quickly identified as having a high likelihood of, or at risk of, developing hemorrhage, i.e., a “vulnerable” patient or hemorrhage-prone patient, when results from pre-PCI coronary angiogram is acquired and/or the computer-implemented simulation is completed. Identifying a “vulnerable” patient provides an informed approach as to how to optimally revascularize the patient in Step ii.

In further embodiments, Step i comprises determining a total score as a summation of a ST-segment score point, an obstruction point, and a collateral point, all before a revascularization therapy is introduced to the subject who is in need thereof, to determine said subject's likelihood (or vulnerability) to develop hemorrhage after revascularization therapy. Hence, if a subject is determined as likely (or vulnerable) to develop hemorrhage, the subject will be administered a gradual reperfusion therapy, as opposed to a standard-rate or rapid reperfusion therapy.

Step ii—Modified Reperfusion:

Various embodiments provide that Step ii includes one of:
(d) Performing a gradual reperfusion to the patient, wherein the patient can be a patient not undergoing any of (a)-(c) of step (i), or an identified vulnerable patient, or an identified non-vulnerable patient; or
(e) Performing a gradual reperfusion to a patient identified as vulnerable (e.g., satisfying at least one, or two, or all three of (a)-(c)) according to step (i), or
   performing a standard rate reperfusion to a patient not identified as vulnerable, or identified as non-vulnerable, according to step (i).

In various embodiments, a gradual reperfusion is administered or effectuated by one or more techniques, including but are not limited to catheter-based reperfusion, and the introduction of a soluble substance into the vascular bed, wherein the soluble substance dissolves over time so as to incrementally increase flow over time. In some embodiments, a soluble substance include ice beads or ice microbeads or other dissolvable beads, such as degradable polymeric beads. In further embodiments, the beads or microbeads contain one or more therapeutic or prophylactic agents, which are released over time.

Non-specific gradual reperfusion—Reperfusion is performed in all patients (i.e., without any details from Step i) gradually (i.e. over a period of time instead of abrupt introduction of blood flow), which would prevent the rupture of small vessels that is central to the development of hemorrhage. A challenge with (d) is that one does not know whether rapid reperfusion would not result in hemorrhage, in which case patient continues to lose heart muscle due to continued ischemia associated with slow reperfusion. On the other hand, the chance of development of hemorrhage is significantly reduced since the pressure within the vessel is not rapidly established so that sudden change in vascular forces don't give away to hemorrhage—this is a benefit for those that would develop hemorrhage but not to those that would not develop hemorrhage even if the artery is rapidly revascularized.

(e) Patient-centric Revascularization—Based on Step i, and the identification of the vulnerable patient, revascularization can be custom tailored to ensure that reperfusion is established without the development of hemorrhage. This would reduce the number of cases that would otherwise be subjected to slow/gradual reperfusion, where hemorrhage would not develop even if the patients were to be revascularized rapidly (note that rapid revascularization is the current standard of care). If a more structured revascularization strategy is adopted, it could take (a) clinical history to identify the vulnerable patient for gradual reperfusion; and/or (b) rely on collateral circulation performed prior to angioplasty; and/or (c) take into account hemodynamic conditions that could help identify the input pressure and duration of reperfusion to minimize or prevent the development of hemorrhage. This is likely to be most successful strategy. In some embodiments, a revascularization treatment method in a subject in need thereof includes: identifying a subject who has all, or any one, two, three, or four of: being a male gender; having left anterior descending coronary artery infarction; having a history of smoking; having prior GPIIb/IIIa infusion; and late presentation after MI (>90 minutes); and performing a gradual reperfusion to the subject. In various aspects, a subject who is a male gender; has left anterior descending coronary artery infarction; has a history of smoking; had prior GPIIb/IIIa infusion; and has late presentation after MI (>90 minutes after the occurrence of heart attack), is vulnerable or likely to develop hemorrhage with standard rapid revascularization therapy. ‘Late presentation’ refers to a late or delayed recognition and seeking of medical attention after the occurrence of a MI, which may involve a delay in recognizing the symptoms of a heart attack, as well as a delay in seeking medical help and being admitted to the hospital for appropriate treatment. In heart attacks, initial 90 minutes are considered golden time period. Beyond that, it is regarded as late presentation.

In various aspects, a patient who may not develop IMH even with rapid revascularization may include all or one or more of: 1. Grade III collaterals; 2. Female gender; 2. Non-LAD infarction 4. No prior thrombolysis or GPIIb/IIIa inhibitor therapy; 5. No history bleeding disorders; and 6. No history of ongoing anticoagulation therapy.

A standard rapid reperfusion typically is characterized in Table 1:

| | Rapid reperfusion |
|---|---|
| TIMI Flow (Grade) | From Grade 0 to III (Max) |
| Pre-dilatation Deflation | From Max Balloon Strength to 0 |
| Pressure (atm) | within 1-2 seconds |
| Lesion Diameter (%) | From 0-100 |
| Distal Pressure 0 | From 0 to Max within 1-2 seconds |

In various embodiments, a gradual reperfusion is characterized in Table 2:

| | Slow/gradual reperfusion |
|---|---|
| TIMI Flow (Grade) | From Grade 0 to I; Or from Grade II to III |
| Pre-dilatation Deflation | From Max Balloon Strength to 0 over 5-10 |
| Pressure (atm) | minutes |
| Culprit artery Diameter (%) | Totally occluded artery is 0. From 0 to 5 |
| | then 10 then 20 then 25 then 50 and then |
| | 100 |
| Distal Pressure (%) | Incremental From 0 to 5 then 10 then 20 |
| | then 25 then 50 and then 100 |

In various implementations, a gradual reperfusion is performed. For example, when the proximal coronary artery is totally occluded, meaning there is a complete blockage of blood flow at the beginning or proximal portion of the coronary artery, the distal pressure downstream from the occlusion site can indeed be very low or close to zero. The goal of the SLOW (also called ‘gradual’) revascularization intervention procedure is to gradually restore the normal diameter of the artery by slowly opening the blockage. With this, eventual slow restoration of blood flow would lead to gradual increase in distal pressure as well in contrast to rapid revascularization where distal bed is exposed to sudden high pressure flow. This as we conceived and demonstrated leads to prevention of hemorrhage by manipulation of pressure-flow.

which increases distal pressure to the catheter from zero (or substantially zero) to levels of the proximal bed over a duration of time. In various aspects, the gradual reperfusion increases the pressure distal to the catheter from zero (or substantially zero) to systolic pressure over a duration of time, wherein the systolic pressure is typically as high as about 100 mmHg in the coronary branches, but also depends on the segment of the coronary branch getting reperfused, hence likely to be below 100 mmHg at peak (e.g., 80-85 mmHg, 85-90 mmHg, 90-95 mmHg, or 95-100 mmHg), and wherein the duration of time for the gradual reperfusion (gradual revascularization intervention) is at least 5 minutes and up to 1 hour. In some aspects, the gradual reperfusion increases the pressure distal to the catheter (or a soluble substance introduced to slowly increase flow rate over time) from zero (or substantially zero) to systolic pressure over at least 10 minutes and up to 1 hour. In some aspects, the gradual reperfusion increases the pressure distal to the catheter (or a soluble substance introduced to slowly increase flow rate over time) from zero (or substantially zero) to systolic pressure over at least 15 minutes and up to 1 hour. In some aspects, the gradual reperfusion increases the pressure distal to the catheter (or a soluble substance introduced to slowly increase flow rate over time) from zero (or substantially zero) to systolic pressure over at least 20 minutes and up to 1 hour. In some aspects, the gradual reperfusion increases the pressure distal to the catheter (or a soluble substance introduced to slowly increase flow rate over time) from zero (or substantially zero) to systolic pressure over at least 30 minutes and up to 1 hour. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 5 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 6 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 7 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 8 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 9 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 10 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 15 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 20 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 25 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 35 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 40 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 45 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 50 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 55 minutes. In some examples, the gradual reperfusion is performed, which increases distal pressure to the catheter (or a soluble substance introduced to slowly increase flow rate over time) from zero (or substantially zero) to 60-65 mmHg, 65-70 mmHg 70-75 mmHg, 75-80 mmHg, 80-85 mmHg, 85-90 mmHg, 90-95 mmHg, or 95-100 mmHg over a time period of at least 60 minutes.

In some examples, the gradual reperfusion is performed, which increases distal pressure to the catheter (or a soluble substance introduced to slowly increase flow rate over time) from a starting pressure to the target pressure at a rate that steadily increases at about 1 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 2 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 3 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 4 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 5 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 6 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 7 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 8 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 9 mmHg per minute. In some examples, the gradual reperfusion is performed, which increases distal pressure to the catheter (or a soluble substance introduced to slowly increase flow rate over time) from a starting pressure to the target pressure at a rate that steadily increases at about 10 mmHg per minute. For example, if the pressure is to steadily increase at a rate of 2 mmHg per minute, over a period of 45 minutes, a pressure starting from 0 mmHg will increase to about 90 mmHg, a pressure starting at about 10 mmHg will increase to about 100 mmHg.

In some examples, the gradual reperfusion is performed, which increases distal pressure to the catheter (or a soluble substance introduced to slowly increase flow rate over time) from a starting pressure to the target pressure, is at variable rate. For example, the first minute of reperfusion achieves a target pressure of about 5 mmHg per, the second minute of reperfusion achieves a target pressure of about 10 mmHg, the third minute of reperfusion achieves a target pressure of about 20 mmHg, the fourth minute achieves a target pressure of about 40 mmHg, and the fourth minute achieves a target pressure of at about 60 mmHg.

This is in contrast to standard rapid-rate reperfusion (or standard-rate revascularization intervention), as a standard-rate vascularization/reperfusion typically initiates without delays other than procedural events preceding the reperfusion and the reperfusion is completed as soon as possible; for example, rapid, immediate, or instantaneous; for example, a standard rapid-rate reperfusion/revascularization intervention is completed within less than 10 minutes, less than 5 minutes, less than 4 minutes, less than 3 minutes, less than 2 minutes, or less than 1 minute. This is also in contrast to reperfusion wherein vascularization/reperfusion is initiated rapidly (e.g., less than 1 minute, less than 2 minutes, less than 5 minutes) to a TIMI grade 3 flow and then allowed to maintain the flow rate for a period of time before stent deployment.

Hence, in some implementations, the duration of time for the gradual reperfusion is at least 50% longer than that for a standard-rate reperfusion. In some implementations, the duration of time for the gradual reperfusion is at least 100% longer than that for a standard-rate reperfusion. In some implementations, the duration of time for the gradual reperfusion is at least 200% longer than that for a standard-rate reperfusion. In some implementations, the duration of time for the gradual reperfusion is at least 300% longer than that for a standard-rate reperfusion.

In additional implementations, a gradual reperfusion (or gradual revascularization intervention) is configured for incrementally opening an occluded blood vessel, e.g., incrementally increasing the size of a balloon tip or a stent in the occluded blood vessel, which may be a step-wise increase or a gradual increase. For example, an incremental enlargement can be an enlargement of about 1%-5% of the previous size or of the original size in about every minute. In another example, an incremental enlargement can be an enlargement of about 1%-5% of the previous size or of the original size in every 2 minutes. In another example, an incremental enlargement can be an enlargement of about 1%-5% of the previous size or of the original size in every 3 minutes. In another example, an incremental enlargement can be an enlargement of about 1%-5% of the previous size or of the original size in every 4 minutes. In another example, an incremental enlargement can be an enlargement of about 1%-5% of the previous size or of the original size in every 5 minutes. In another example, an incremental enlargement can be an enlargement of about 1%-5% of the previous size or of the original size in every 10 minutes. In another example, an incremental enlargement can be an enlargement of about 5%-10% of the previous size or of the original size in about every minute. In another example, an incremental enlargement can be an enlargement of about 5%-10% of the previous size or of the original size in about every 2 minutes. In another example, an incremental enlargement can be an enlargement of about 5%-10% of the previous size or of the original size in about every 3 minutes. In another example, an incremental enlargement can be an enlargement of about 5%-10% of the previous size or of the original size in about every 4 minutes. In another example, an incremental enlargement can be an enlargement of about 5%-10% of the previous size or of the original size in about every 5 minutes. In another example, an incremental enlargement can be an enlargement of about 5%-10% of the previous size or of the original size in about every 10 minutes. Yet in another example, an incremental enlargement can be an enlargement of about 10%-20% of the previous size or of the original size in about every minute. In another example, an incremental enlargement can be an enlargement of about 10%-20% of the previous size or of the original size in about every 5 minutes. In another example, an incremental enlargement can be an enlargement of about 10%-20% of the previous size or of the original size in about every 10 minutes. In a further example, an incremental enlargement results in full lumen patency for a blood vessel over a period of time, for example, steadily over a period of at least 10 minutes, but preferably no more than 1 hour. In a further example, an incremental enlargement results in full lumen patency for a blood vessel over a period of time, for example, steadily over a period of at least 20 minutes, but preferably no more than 1 hour. In a further example, an incremental enlargement results in full lumen patency for a blood vessel over a period of time, for example, steadily over a period of at least 30 minutes; but preferably no more than 1 hour.

Various embodiments provide methods for treating a subject with myocardial infarction (MI), or performing revascularization in a subject with MI, and the methods include: (i) identifying a subject with a likelihood to develop intramyocardial hemorrhage, and (ii) performing a gradual revascularization intervention in the subject with the MI who is identified with the likelihood (or vulnerability) to develop intramyocardial hemorrhage, wherein the gradual revascularization intervention is slower or takes longer in restoring myocardial reperfusion compared to the standard-rate vascularization intervention.

Other embodiments provide methods for treating a subject with myocardial infarction (MI), or performing revascularization in a subject with MI, wherein the methods include performing a standard rapid-rate revascularization intervention to a subject with the MI who is identified as not having a likelihood (or having a reduced likelihood) to develop intramyocardial hemorrhage.

Hence several embodiments provide methods for treating a subject with myocardial infarction (MI), or performing revascularization in a subject with MI, wherein the methods include performing a gradual revascularization intervention to a subject identified with a likelihood to develop intramyocardial hemorrhage, or performing a standard-rate revascularization intervention to a subject identified as not having the likelihood (or lower likelihood) to develop intramyocardial hemorrhage.

Yet additional embodiments provide methods for treating a subject with myocardial infarction (MI), or performing revascularization in a subject with MI, and the methods include performing a gradual revascularization intervention in the subject with the MI, regardless of whether or not the subject is examined for, or identified with, a likelihood to develop intramyocardial hemorrhage.

In some embodiments, a subject is identified with a likelihood to develop intramyocardial hemorrhage by:
(a) determining or obtaining results that the subject has an ongoing inflammation-related disorder, diabetes, hypertension, a metabolic disease, or a combination thereof, or that the subject with MI has had the diabetes, the hypertension, the metabolic disease, or a combination thereof prior to the MI;
(b) performing, or obtaining or requesting results of, an angiogram in the subject to determine absence of collateral coronary circulation in the subject; or
(c) performing, or obtaining or requesting results of, a computer-implemented simulation on one or more hemodynamic parameters of an ischemic artery of the subject to determine presence of a likelihood of an undesirable abrupt change in the hemodynamic parameters when a standard-rate revascularization intervention is simulated;
or a combination of (a)-(c).

In some aspects, a subject is identified with a likelihood to develop intramyocardial hemorrhage by determining or obtaining results that the subject has an ongoing inflammation-related disorder, diabetes, hypertension, a metabolic disease, or a combination thereof, or that the subject with MI has had the diabetes, the hypertension, the metabolic disease, or a combination thereof prior to the MI.

In some aspects, a subject is identified with a likelihood to develop intramyocardial hemorrhage by performing, or obtaining or requesting results of, an angiogram in the subject to determine absence of collateral coronary circulation in the subject

In some aspects, a subject is identified with a likelihood to develop intramyocardial hemorrhage by performing, or obtaining or requesting results of, a computer-implemented simulation on one or more hemodynamic parameters of an ischemic artery of the subject to determine presence of a likelihood of an undesirable abrupt change in the hemodynamic parameters when a standard-rate revascularization intervention is simulated.

In some aspects, a subject is identified with a likelihood to develop intramyocardial hemorrhage by a combination of (a)-(c).

In some embodiments, a subject is identified as not having a likelihood (or lower likelihood) to develop intramyocardial hemorrhage by:
determining or obtaining results that the subject does not have an ongoing inflammation-related disorder, diabetes, hypertension, or a metabolic disease, and that the subject did not have diabetes, hypertension, or a metabolic disease prior to the MI;
performing, or obtaining or requesting results of, an angiogram in the subject to determine presence of collateral coronary circulation in the subject; and/or
performing, or obtaining or requesting results of, a computer-implemented simulation on one or more hemodynamic parameters of an ischemic artery of the subject to determine absence of a likelihood (or lower likelihood) of an undesirable abrupt change in the hemodynamic parameters when the standard-rate revascularization intervention is simulated.

In some aspects, a subject is identified as not having a likelihood (or lower likelihood) to develop intramyocardial hemorrhage by: determining or obtaining results that the subject does not have an ongoing inflammation-related disorder, diabetes, hypertension, or a metabolic disease, and that the subject did not have diabetes, hypertension, or a metabolic disease prior to the MI; performing, or obtaining or requesting results of, an angiogram in the subject to determine presence of collateral coronary circulation in the subject; AND performing, or obtaining or requesting results of, a total score of ST-segment score point, collateral point, and occlusion point of the subject to determine unlikelihood or reduced likelihood of developing hemorrhage with standard rapid-rate revascularization intervention.

An interventional device or a system that can be employed in the setting of reperfusion therapy, for patients having myocardial infarction. For example, instruments (such as soluble substance that dissolves over time when placed in blood vessel bed) and/or stents which are adapted for gradual reperfusion and/or patient-centric (patient customized) revascularization regimen.

In some embodiments, the systems include a device and a software, wherein the software provides instructions to operate the device such that the device revascularizes a blood vessel of a patient in an incremental enlargement fashion prior to fully opening the vessel. In some embodiments, the device includes a catheter and optionally further a stent, and the device is configured to operate (under instructions provided by the software) such that the hydrostatic pressure transmitted from the proximal end of the catheter is modulated, prior to placing a stent. In some embodiments, the device includes a stent, and the device is configured to be placed within a central core of an embolization of a patient and to be operated (under instructions provided by the software) to slowly expand within a vessel lumen over a period of time, so as to incrementally enlarge blood flow in the vessel.

Some embodiments provide for a catheter modulation system for performing a gradual revascularization intervention to a patient, which includes one or more or all of:
a catheter adapted to be inserted into a blood vessel of the patient,
a balloon tip connected to the catheter, optionally the balloon tip being covered with a stent,
a balloon tip inflation mechanism for introducing fluid through the catheter and into the balloon tip at a predetermined rate to inflate the balloon tip, and
a controller for controlling the balloon tip inflation mechanism, wherein the controller controls amount of fluid to be introduced into the balloon tip,
wherein the predetermined rate to inflate the balloon tip is one effective for increasing a pressure distal to the balloon tip from substantially zero to systolic pressure of the patient over a duration of at least 5 minutes, at least 10 minutes, or at least 30 minutes and up to 1 hour, or wherein the predetermined rate to inflate the balloon tip is one effective for increasing a pressure distal to the balloon tip from substantially zero to systolic pressure, or from a starting pressure to a systolic pressure, of the patient over a duration that is at least 100%, at least 200%, or at least 300% longer than that for a standard-rate revascularization intervention.

In some aspects, the catheter modulation system may further include one or more or all of:
a memory storing a set of patient-specific, predetermined rates and/or amounts of fluid to be introduced to inflate the balloon tip;
a data acquiring routine for obtaining data pertaining to blood pressure or blood flow near the balloon tip; and/or
a control routine for processing the data pertaining to the blood pressure or blood flow, and a current size of the balloon tip or a current rate and amount of introduced fluid into the balloon tip and for automatically changing the rate and amount of the fluid to be introduced into the balloon tip in accordance with the set of patient-specific, predetermined rates and/or amounts of fluid.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Cardioprotection from Collateralized Coronary Vascular Beds in Hemorrhagic ST-Elevation Myocardial Infarction

Annual incidence of acute myocardial infarction is approximately 1 million in the United States alone. ‘Time is Muscle’ is the principle around emergency coronary angiography (CAG) and percutaneous coronary intervention (PCI) to reduce ischemic injury among these patients. However, intramyocardial hemorrhage (IMH) has emerged as a critical component of ischemia-reperfusion injury following reperfusion therapy. Cardiac magnetic resonance (CMR) is the gold standard technique to identify IMH upon stabilization of the infarct. Though it is impractical to perform CMR in every post-intervention patient to identify IMH. Therefore, it is very important to stratify the right AMI patient population to detect IMH. We have identified angiographic detection of coronary collaterals in AMI population as an important cardioprotection against IMH. Absence of coronary collaterals is an important predictive risk factor for post-reperfusion IMH.

ST-elevation myocardial infarction (STEMI) patients (n=80) who underwent emergency CAG and PCI were identified for grading of coronary collaterals based on Rentop Classification. Cardiac MRI (CMR) was performed 3 days post PCI with LGE and T2* parametric imaging to determine the presence and extent of hemorrhage volume, and infarct size.

Among 80 patients, IMH was observed in 58 (72.5%) patients, and 22 (27.5%) patients were non-hemorrhagic. 55.17% of hemorrhagic and 13.63% of non-hemorrhagic MI patients had grade 0 collaterals respectively; while 44.48% and 86.36% had grade I/II/III collaterals respectively. Collaterals were absent (Grade 0) in 35 patients; of these, 32 (91%) had IMH. Collaterals were present (Grade I/II/III) in 45 patients; of these, 58% had IMH. 55.17% of hemorrhagic and 13.63% of non-hemorrhagic MI patients had grade 0 collaterals respectively while 44.48% and 86.36% had grade I/II/III collaterals respectively. Compared to those with collaterals, those without collaterals had 12% larger MI and 37% larger IMH volume (p<0.01, both). That is, 55.17% of hemorrhagic and 13.63% of non-hemorrhagic MI patients had grade 0 collaterals respectively while 44.48% and 86.36% had grade I/II/III collaterals respectively.

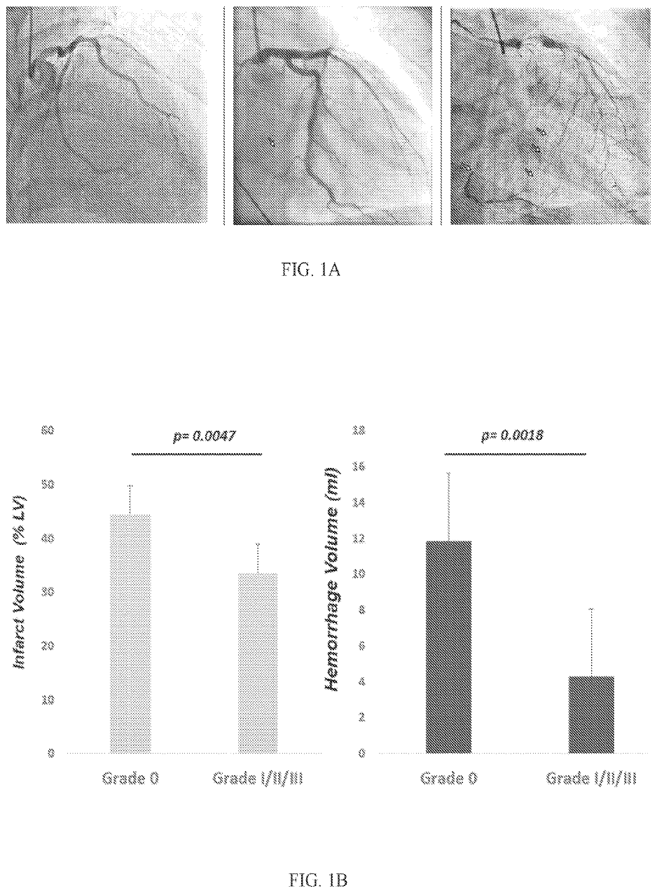

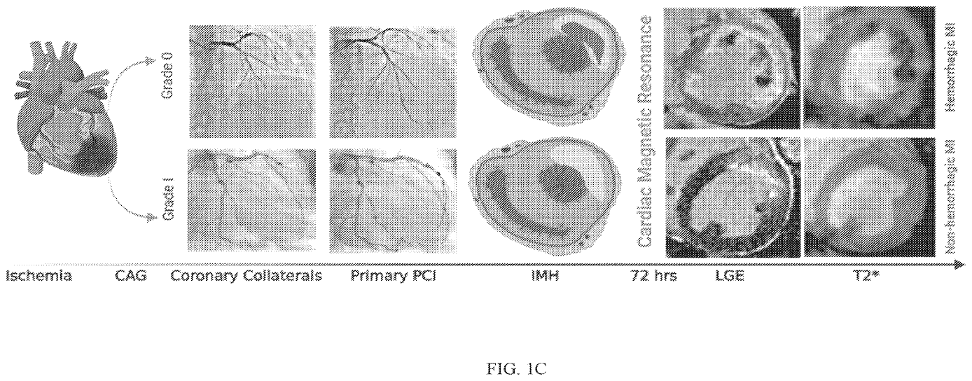

FIG. 1A shows representative cases of pre-PCI CAG images for collateral grading by Rentrop's Classification. Grade 0=No visible filling of any collateral channels; Grade I=Collateral filling of branches of the vessel without any dye reaching the epicardial segment of that vessel; Grade II=Partial collateral filling of the epicardial segment of the vessel.

FIG. 1B (left) shows infarct size of hemorrhagic MI cases with and without collaterals. Infarct size is calculated by late gadolinium enhancement (LGE) area on CMR. In the presence of collaterals, infarct size is 11.77% (p value: 0.006) lower than absence of collaterals. FIG. 1B (right) shows hemorrhage volume in hemorrhagic MI cases with and without collaterals. Hemorrhage volume is calculated by T2* parametric imaging on CMR. In the presence of collaterals, hemorrhage volume is 36.94% (p-value: 0.0018) lower than absence of collaterals.

Collateralized coronary beds reduce the incidence and extent of IMH and MI size in STEMI, and as such they appear to be cardioprotective against IMH. In STEMI patients, coronary collaterals reduced IMH incidence by 41.88% in post-reperfusion period confirmed by CMR. Collaterals reduce IMH volume by 36.94% as compared to the Grade 0 collateral group among the hemorrhagic MI patient population. This concludes that coronary collaterals are important in-built cardioprotection against IMH-mediated ischemia-reperfusion injury and CMR can be a modality of choice for IMH evaluation in AMI patients without coronary collaterals.

### Example 2. New Explainable AI Method Reveals a Simplified Model that Accurately Predicts Post-Reperfusion Intramyocardial Hemorrhage Using Three Readily Available Clinical Variables

Myocardial infarction (MI) is a major global health issue. A critical complication for patients undergoing primary percutaneous coronary intervention (PCI) is post-reperfusion intramyocardial hemorrhage (IMH), contributing to impaired left ventricular function, adverse remodeling, chronic heart failure, and increased mortality rates. Cardiac magnetic resonance (CMR), the gold standard for IMH identification, faces limitations in time constraints, availability, and contraindications for some individuals. Early identification of high-risk patients enables personalized clinical management and targeted CMR diagnosis and can enhance patient outcomes, allowing aggressive management of comorbidities such as hypertension and diabetes.

Artificial intelligence (AI) holds the potential to develop predictive models for IMH by leveraging clinical, demographic, and procedural factors. Nonetheless, accuracy of such models is generally contingent upon access to substantial data. Moreover, they often function as black boxes without a clear understanding of the underlying rationale behind predictions. This poses a significant barrier for their adoption in critical clinical settings.

Advancements in explainable AI have led to an innovative additive deep learning architecture called superposable neural networks (SNN). By identifying features and their interdependencies upfront and incorporating them as independent inputs, SNNs offer full transparency and enable incorporating expert knowledge.

Herein we have created a simplified predictive model that accurately predicts post-reperfusion IMH using readily available clinical information.

A total of 310 STEMI patients who undergone PCI were enrolled in the study. Patients underwent CMR with T2* mapping within 48-72 hours post-PCI for IMH ground-truth. 12-lead ECGs were analyzed for ST-segment scores. The catheterization angiography was assessed for collateral status, identifying the culprit artery and lesion degree and location.

The SNN method was used to determine the minimum features required for predicting IMH without sacrificing accuracy. Expert knowledge was utilized to fill the gap in missing ST-segment score data by fitting an exponential curve to the function produced by the SNN.

Three features were identified as sufficient for predicting IMH: collaterals-presence, lesion-degree, and ST-segment score, with 81.4% accuracy, 83.5% sensitivity, and 78.5% specificity.

We presented a simplified highly-convenient predictive model for early detection of IMH risk, that can enable personalized clinical management and targeted CMR diagnosis and enhance patient outcomes.

### Example 3. Gradual Reperfusion Reduces Volume of Intramyocardial Hemorrhage in Dogs

Animal Model: Dogs (20-25 kg, n=4) at least 6 months-old were fasted for 18 hours prior to the surgical procedure. Canines received Carprofen (4.0 mg/kg of body weight, per os (by mouth)) on the morning of the surgical procedure and received antibiotics prior to the surgery (Keflex, 1000 mg/kg of body weight, per os). Each dog was sedated using acepromazine (0.5-1.0 mg/kg body weight, subcutaneous) or Midazolam (0.4 mg/kg body weight, intramuscular) to set up the intravenous line. Subsequently, the dog were anesthetized with an intravenous injection of Propofol (5.0-7.5 mg/kg), endotracheally intubated and maintained on gas anesthesia (2.0-2.5% isoflurane with 100% oxygen). Animals were artificially ventilated at 1-2 L/min with the respiration rate being continuously adjusted to maintain partial pressure of CO₂ in blood (PaCO₂) between 30 and 35 mmHg. To minimize fatal ventricular arrhythmias from myocardial infarction (MI), all dogs were pre-treated with Amiodarone (200 mg/day, TEVA Pharmaceuticals USA, Sellersville, PA, USA) for 2 weeks prior to inducing MI. Left lateral thoracotomy was performed at the fourth intercostal space, and the exposed heart was suspended in a pericardial cradle. Aortic and left atrial catheters were inserted and secured for invasive blood pressure monitoring and drug delivery. A portion of the left anterior descending (LAD) artery was isolated just past the diagonal or marginal branches, and a hydraulic occlude be looped around the vessel (with a doppler flow probe affixed distal to the occlude to assess blow flow). Animals' chest walls were then closed, skins sutured and they were allowed to recover for 2 weeks. Subsequently, LAD infarcts were created within side the MRI scanner. LAD was ligated for three hours and then reperfused immediately in two animals; the other two animals were reperfused gradually (approximately 30% of baseline flow first 20 mins, approximately 60% of baseline flow the next 20 mins, and complete release thereafter). Doppler flow probe being utilized to assess the absence/presence of flow following ligation.

Cardiac MRI (CMR) Data Acquisition: We performed multiple, breath-held, contiguous, short-axis 2D cine, T2*-weighted and LGE images triggered at mid-diastole and covering the full LV following whole-heart shimming and scout scans 3 days post infarction. Cine images were acquired using bSSFP (TR/TE=1.0/1.5 ms, flip angle (FA)=50°, with at least 24 cardiac phases at a minimum temporal resolution of 50 ms). T2* maps (Aim 1) were constructed on the basis of multi-gradient echo T2*-weighted images (TR=12 ms, 10 TEs=1.1 ms-11.0 ms with ΔTE=1.1 ms, FA=10°); LGE images were acquired using an inversion-recovery prepared FLASH (TR/TE=3.0/1.5 ms, FA=25° in the standard manner (10-15 minutes after 0.2 mmol/Kg of Gd (Gadovist). Spatial resolution of all sequences were set to 1.5×1.5×5 mm³.

CMR Analysis: MI size, hemorrhage volume and corresponding concentration (R2*), were quantified using CVI (Circle Inc., Canada). Briefly, endo- and epi-cardial contours were drawn to segment the myocardium. The remote myocardium was identified on LGE images showing no hyperintensity were used as a reference region-of-interest (ROI) on both LGE and T2* images. Subsequently, all MI size, hemorrhage volume and the corresponding R2* were segmented and quantified automatically in CVI. Infarct size was defined as the hyperintense region on LGE images with mean signal intensity >5 standard deviations (SD) above that of the reference ROI. Hypointense regions within the LGE enhancement territory, which do not conform to the mean signal intensity >5 SD were labeled as persistent microvascular obstruction and their volume within an imaging slice were computed by subtracting the territory of LGE with signal intensity >5SD from territory of the LGE obtained by including the MVO. Hemorrhage was identified and quantified as the infarcted territory containing hypointense values that are <2SD of the reference ROI. MI size (% LV) as computed by summing the slice measures and normalizing by the myocardial LV volume. Hemorrhage volume (% LV) was computed by dividing the hypointense volume identified on the slice basis, summed over all the slices positive for hemorrhage and then normalizing by the myocardial LV volume.

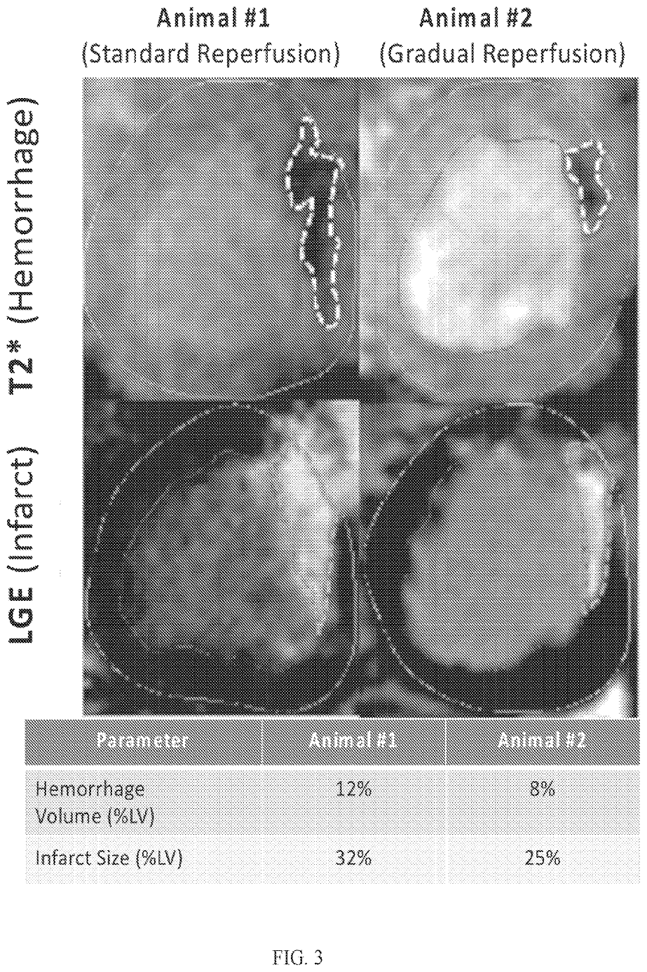

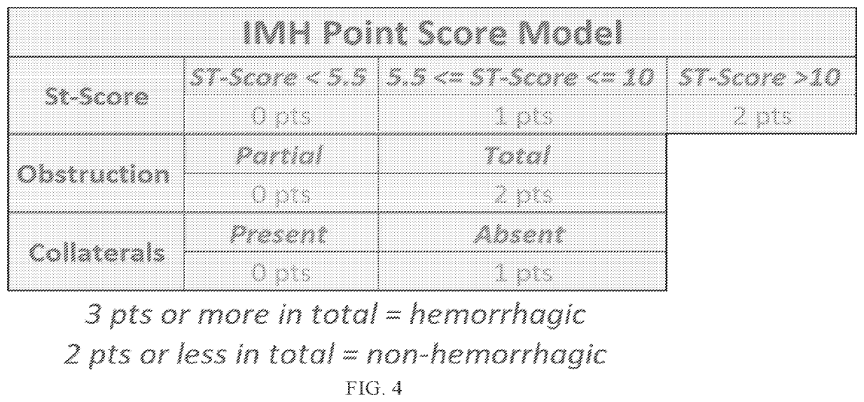

FIG. 3 shows representative findings from the study which demonstrates that gradual reperfusion can lead to reduced hemorrhage volume. Reduction in hemorrhage volume is also associated with reduced infarct size. Aggregate data across animals showed that in animals undergoing standard reperfusion results in a MI size of 29%+/−4% and hemorrhage volume of 10%+/−3%, compared to MI size of 21%+/−6% and hemorrhage volume of 7%+/−1% in those that were reperfused in a gradual manner.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method for treating a subject with myocardial infarction (MI), or performing revascularization in a subject with MI, the subject with the MI having at least one occluded coronary blood vessel causing the acute MI, the method comprising:
(i) identifying a subject with a likelihood to develop intramyocardial hemorrhage by performing step (c), or any one or more of (a)-(c), below:
(a) determining or obtaining results that the subject has an ongoing inflammation-related disorder, diabetes, hypertension, a metabolic disease, or a combination thereof, or that the subject with MI has had the diabetes, the hypertension, the metabolic disease, or a combination thereof prior to the MI;
(b) performing, or obtaining or requesting results of, an angiogram in the subject to determine absence of collateral coronary circulation in the subject; or
(c) performing, or obtaining or requesting results of, a full occlusion of an ischemic artery, AND, a ST-segment score of at least 5.5 and/or an absence of collateral coronary circulation of the ischemic artery; OR performing, or obtaining or requesting results that the occlusion of the ischemic artery is only partial and not full occlusion, the ST-segment score is greater than 10, and the collateral coronary circulation of the ischemic artery is present, so as to determine increased likelihood of intramyocardial hemorrhage following a standard-rate revascularization intervention;
or a combination of (a)-(c);
and
(ii) performing a gradual revascularization intervention in the subject with the MI who is identified with the likelihood to develop intramyocardial hemorrhage, wherein the gradual revascularization intervention is slower or takes longer in restoring myocardial reperfusion compared to the standard-rate vascularization intervention.

2. A method for treating a subject with myocardial infarction (MI), or performing revascularization in a subject with MI, the subject with the MI having at least one occluded coronary blood vessel causing the acute MI, the method comprising:
performing a standard-rate revascularization intervention to a subject with the MI who is identified as not having a likelihood or lower likelihood to develop intramyocardial hemorrhage,
wherein the subject not having the likelihood or lower likelihood is identified by:
determining or obtaining results that the subject does not have an ongoing inflammation-related disorder, diabetes, hypertension, or a metabolic disease, and that the subject did not have diabetes, hypertension, or a metabolic disease prior to the MI;
performing, or obtaining or requesting results of, an angiogram in the subject to determine presence of collateral coronary circulation in the subject; and/or
performing, or obtaining or requesting results of, a partial occlusion of an ischemic artery of the subject and a presence of collateral coronary circulation of the ischemic artery, OR a partial occlusion of the ischemic artery with an absence of the collateral coronary circulation and a ST-segment score of 10 or less, OR a total occlusion of the ischemic artery with a presence of the collateral coronary circulation and a ST-segment score of less than 5.5, to determine an unlikelihood of intramyocardial hemorrhage following a standard-rate revascularization intervention.

3. A method for treating a subject with myocardial infarction (MI), or performing revascularization in a subject with MI, the subject with the MI having at least one occluded coronary blood vessel causing the acute MI, the method comprising:
performing a gradual revascularization intervention in the subject with the MI, wherein the gradual revascularization intervention is slower or takes longer in restoring myocardial reperfusion compared to a standard-rate vascularization intervention.

4. The method of claim 3, wherein the subject did not undergo an angiogram prior to receiving the gradual revascularization intervention, or wherein the method does not include performing, or obtaining or requesting results of, an angiogram of the subject, prior to performing the gradual revascularization intervention in the subject.

5. The method of claim 3, wherein the method does not include requesting or obtaining results from the subject of an ongoing or prior existence of diabetes, hypertension, a metabolic disease, or an inflammation-related disorder, prior to performing the gradual revascularization intervention in the subject.

6. The method of claim 3, wherein the method does not include performing, or obtaining or requesting results of, a computer-implemented simulation on one or more hemodynamic parameters of an ischemic artery of the subject prior to performing the gradual revascularization intervention in the subject.

7. The method of claim 3, wherein the subject with the MI is identified with a likelihood to develop intramyocardial hemorrhage by:
(a) determining or obtaining results that the subject has an ongoing inflammation-related disorder, diabetes, hypertension, a metabolic disease, or a combination thereof, or that the subject with MI has had the diabetes, the hypertension, the metabolic disease, or a combination thereof prior to the MI;
(b) performing, or obtaining or requesting results of, an angiogram in the subject to determine absence of collateral coronary circulation in the subject; or
(c) performing, or obtaining or requesting results of, a ST-segment score of greater than 10 from electrocardiography, OR full occlusion of an ischemic artery of the subject, OR an absence of collateral coronary circulation of the ischemic artery with a ST-segment score of at least 5.5, to determine likelihood of intramyocardial hemorrhage following a standard-rate revascularization intervention;
or a combination of (a)-(c).

8. The method of claim 1, wherein said revascularization intervention comprises percutaneous coronary intervention, intracoronary stent, or atherectomy device.

9. The method of claim 1, wherein the standard-rate revascularization intervention comprises a percutaneous coronary intervention or a cardiac catheterization configured to increase pressure distal to a catheter from substantially zero to systolic pressure, or from a starting pressure to a systolic pressure, of the subject over a duration of no more than 5 minutes or no more than 10 minutes.

10. The method of claim 1, wherein the gradual revascularization intervention comprises a percutaneous coronary intervention or a cardiac catheterization configured to increase pressure distal to a catheter from substantially zero to systolic pressure, or from a starting pressure to a systolic pressure, of the subject over a duration of at least 15 minutes, at least 30 minutes, at least 40 minutes or at least 50 minutes and up to 1 hour.

11. The method of claim 1, wherein the gradual revascularization intervention comprises a percutaneous coronary intervention or a cardiac catheterization configured to increase pressure distal to a catheter from substantially zero to systolic pressure, or from a starting pressure to a systolic pressure, of the subject over a duration that is at least 100%, at least 200%, or at least 300% longer than that for a standard-rate revascularization intervention.

12. The method of claim 1, wherein performing the angiogram further comprises segmenting blood vessels from the angiogram to determine absence or presence of collateral circulation in each segment of the blood vessels.

13. The method of claim 1, further comprising calculating a total score of an assigned ST-segment score point, an assigned occlusion point, and an assigned collateral point of the subject, wherein the total score is higher compared to that of a reference individual who did develop hemorrhage after a standard-rate revascularization therapy, wherein the point assignment includes a higher point for a higher ST-segment score relative to a lower ST-segment score, a higher point for full occlusion relative to partial occlusion, and a higher point for absence of collateral coronary circulation relative to presence of the collateral coronary circulation, for a same individual.

14. The method of claim 2, further comprising calculating a total score of an assigned ST-segment score point, an assigned occlusion point, and an assigned collateral point of the subject, wherein the total score is lower compared to that of a reference individual who did not develop hemorrhage after the standard-rate revascularization therapy, wherein the point assignment includes a higher point for a higher ST-segment score relative to a lower ST-segment score, a higher point for full occlusion relative to partial occlusion, and a higher point for absence of collateral coronary circulation relative to presence of the collateral coronary circulation, for a same individual.

15. The method of claim 1, wherein the method restores TIMI grade 3 flow in the occluded coronary blood vessel in the subject.

16. A method for assessing a subject in need of a coronary revascularization therapy, and/or prognosing the likelihood of developing intramyocardial hemorrhage following a standard-rate coronary revascularization therapy in the subject, the method comprising:
performing an electrocardiogram in the subject to obtain ST-segment score, and performing a coronary angiogram in the subject to determine presence or absence of collateral coronary circulation and extent of culprit artery obstruction;
and
determining the subject is likely to develop intramyocardial hemorrhage following the standard-rate coronary revascularization therapy, if:
(1) the extent of culprit artery obstruction is full occlusion of an ischemic artery, AND, the ST-segment score is at least 5.5 and/or the collateral coronary circulation of the ischemic artery is absent; or
the extent of culprit artery obstruction is only partial occlusion of the ischemic artery, AND, the ST-segment score is greater than 10 and the collateral coronary circulation of the ischemic artery is absent; or
(2) a total score of an assigned ST-segment score point, an assigned occlusion point, and an assigned collateral point, is higher than that of a reference individual who did develop hemorrhage after the standard-rate revascularization therapy;
or determining the subject is unlikely or has a reduced likelihood to develop intramyocardial hemorrhage following the standard-rate coronary revascularization therapy, if:
(3) the extent of culprit artery occlusion is only partial occlusion and the collateral coronary circulation is present; or
the extent of culprit artery occlusion is only partial occlusion, the collateral coronary circulation is absent, and the ST-segment score is 10 or less; or
the extent of culprit artery occlusion is full occlusion of the ischemic artery, the collateral coronary circulation is present, and the ST-segment score is less than 5.5; or
(4) the total score of the assigned ST-segment score point, the assigned occlusion point, and the assigned collateral point, is lower than that of a reference individual who did not develop hemorrhage after the standard-rate revascularization therapy;
wherein the point assignment includes a higher point for a higher ST-segment score relative to a lower ST-segment score, a higher point for full occlusion relative to partial occlusion, and a higher point for absence of collateral coronary circulation relative to presence of the collateral coronary circulation, for a same individual.

17. The method of claim 16, further comprising prescribing or administering the standard-rate revascularization therapy to the subject determined to have an unlikelihood or reduced likelihood of developing the hemorrhage.

18. The method of claim 16, further comprising prescribing or administering a gradual revascularization intervention to the subject determined to have an likelihood of developing the hemorrhage.
